SEQUENCE LISTING (7 sequences)

SEQ ID NO: 1 - DNA, 8035 bp, Escherichia coli
tctagcaggactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcctccgcggagcgctcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctgtgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttgtggataatgttttttgcgccgacatcataacggttctggcaaatattctgaaatgagctgttgacaattaatcatccggctcgtataatgtgtggaattgtgagcggataacaatttcacacaggaaacagaccatggcacaccatcaccaccatcacgcctatccatacgatgtgccagattatgcaatggtttactcctataccgagaaaaaacgtattcgtaaggattttggtaaacgtccacaagttctggatgtaccttatctcctttctatccagcttgactcgtttcagaaatttatcgagcaagatcctgaagggcagtatggtctggaagctgctttccgttccgtattcccgattcagagctacagcggtaattccgagctgcaatacgtcagctaccgccttggcgaaccggtgtttgacgtccaggaatgtcaaatccgtggcgtgacctattccgcaccgctgcgcgttaaactgcgtctggtgatctatgagcgcgaagcgccggaaggcaccgtaaaagacattaaagaacaagaagtctacatgggcgaaattccgctcatgacagacaacggtacctttgttatcaacggtactgagcgtgttatcgtttcccagctgcaccgtagtccgggcgtcttctttgactccgacaaaggtaaaacccactcttcgggtaaagtgctgtataacgcgcgtatcatcccttaccgtggttcctggctggacttcgaattcgatccgaaggacaacctgttcgtacgtatcgaccgtcgccgtaaactgcctgcgaccatcattctgcgcgccctgaactacaccacagagcagatcctcgacctgttctttgaaaaagttatctttgaaatccgtgataacaagctgcagatggaactggtgccggaacgcctgcgtggtgaaaccgcatcttttgacatcgaagctaacggtaaagtgtacgtagaaaaaggccgccgtatcactgcgcgccacattcgccagctggaaaaagacgacgtcaaactgatcgaagtcccggttgagtacatcgcaggtaaagtggttgctaaagactatattgatgagtctaccggcgagctgatctgcgcagcgaacatggagctgagcctggatctgctggctaagctgagccagtctggtcacaagcgtatcgaaacgctgttcaccaacgatctggatcacggcccatatatctctgaaaccttacgtgtcgacccaactaacgaccgtctgagcgcactggtagaaatctaccgcatgatgcgccctggcgagccgccgactcgtgaagcagctgaaagcctgttcgagaacctgttcttctccgaagaccgttatgacttgtctgcggttggtcgtatgaagttcaaccgttctctgctgcgcgaagaaatcgaaggttccggtatcctgagcaaagacgacatcattgatgttatgaaaaagctcatcgatatccgtaacggtaaaggcgaagtcgatgatatcgaccacctcggcaaccgtcgtatccgttccgttggcgaaatggcggaaaaccagttccgcgttggcctggtacgtgtagagcgtgcggtgaaagagcgtctgtctctgggcgatctggataccctgatgccacaggatatgatcaacgccaagccgatttccgcagcagtgaaagagttcttcggttccagccagctgtctcagtttatggaccagaacaacccgctgtctgagattacgcacaaacgtcgtatcttcgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgaatacggcttccttgagactccgtatcgtaaagtgaccgacggtgttgtaactgacgaaattcactacctgtctgctatcgaagaaggcaactacgttatcgcccaggcgaactccaacttggatgaagaaggccacttcgtagaagacctggtaacttgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggccgttccgactctgcgcgctgataagccgctggttggtactggtatggaacgtgctgttgccgttgactccggtgtaactgcggtagctaaacgtggtggtgtcgttcagtacgtggatgcttcccgtatcgttatcaaagttaacgaagacgagatgtatccgggtgaagcaggtatcgacatctacaacctgaccaaatacacccgttctaaccagaacacctgtatcaaccagatgccgtgtgtgtctctgggtgaaccggttgaacgtggcgacgtgctggcagacggtccgtccaccgacctcggtgaactggcgcttggtcagaacatgcgcgtagcgttcatgccgtggaatggttacaacttcgaagactccatcctcgtatccgagcgtgttgttcaggaagaccgtttcaccaccatccacattcaggaactggcgtgtgtgtcccgtgacaccaagctgggtccggaagagatcaccgctgacatcccgaacgtgggtgaagctgcgctctccaaactggatgaatccggtatcgtttacattggtgcggaagtgaccggtggcgacattctggttggtaaggtaacgccgaaaggtgaaactcagctgaccccagaagaaaaactgctgcgtgcgatcttcggtgagaaagcctctgacgttaaagactcttctctgcgcgtaccaaacggtgtatccggtacggttatcgacgttcaggtctttactcgcgatggcgtagaaaaagacaaacgtgcgctggaaatcgaagaaatgcagctcaaacaggcgaagaaagacctgtctgaagaactgcagatcctcgaagcgggtctgttcagccgtatccgtgctgtgctggtagccggtggcgttgaagctgagaagctcgacaaactgccgcgcgatcgctggctggagctgggcctgacagacgaagagaaacaaaatcagctggaacagctggctgagcagtatgacgaactgaaacacgagttcgagaagaaactcgaagcgaaacgccgcaaaatcacccagggcgacgatctggcaccgggcgtgctgaagattgttaaggtatatctggcggttaaacgccgtatccagcctggtgacaagatggcaggtcgtcacggtaacaagggtgtaatttctaagatcaacccgatcgaagatatgccttacgatgaaaacggtacgccggtagacatcgtactgaacccgctgggcgtaccgtctcgtatgaacatcggtcagatcctcgaaacccacctgggtatggctgcgaaaggtatcggcgacaagatcaacgccatgctgaaacagcagcaagaagtcgcgaaactgcgcgaattcatccagcgtgcgtacgatctgggcgctgacgttcgtcagaaagttgacctgagtaccttcagcgatgaagaagttatgcgtctggctgaaaacctgcgcaaaggtatgccaatcgcaacgccggtgttcgacggtgcgaaagaagcagaaattaaagagctgctgaaacttggcgacctgccgacttccggtcagatccgcctgtacgatggtcgcactggtgaacagttcgagcgtccggtaaccgttggttacatgtacatgctgaaactgaaccacctggtcgacgacaagatgcacgcgcgttccaccggttcttacagcctggttactcagcagccgctgggtggtaaggcacagttcggtggtcagcgtttcggggagatggaagtgtgggcgctggaagcatacggcgcagcatacaccctgcaggaaatgctcaccgttaagtctgatgacgtgaacggtcgtaccaagatgtataaaaacatcgtggacggcaaccatcagatggagccgggcatgccagaatccttcaacgtattgttgaaagagattcgttcgctgggtatcaacatcgaactggaagacgagtaattctcgctcaaacaggtcactgctgtcgggttaaaacccgaccgcagcccaagctgggtaccgagctcggtacccgatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaa

SEQ ID NO: 2 - DNA, 8035 bp, Escherichia coli
tctagcaggactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcctccgcggagcgctcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctgtgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttgtggataatgttttttgcgccgacatcataacggttctggcaaatattctgaaatgagctgttgacaattaatcatccggctcgtataatgtgtggaattgtgagcggataacaatttcacacaggaaacagaccatggcacaccatcaccaccatcacgcctatccatacgatgtgccagattatgcaatggtttactcctataccgagaaaaaacgtattcgtaaggattttggtaaacgtccacaagttctggatgtaccttatctcctttctatccagcttgactcgtttcagaaatttatcgagcaagatcctgaagggcagtatggtctggaagctgctttccgttccgtattcccgattcagagctacagcggtaattccgagctgcaatacgtcagctaccgccttggcgaaccggtgtttgacgtccaggaatgtcaaatccgtggcgtgacctattccgcaccgctgcgcgttaaactgcgtctggtgatctatgagcgcgaagcgccggaaggcaccgtaaaagacattaaagaacaagaagtctacatgggcgaaattccgctcatgacagacaacggtacctttgttatcaacggtactgagcgtgttatcgtttcccagctgcaccgtagtccgggcgtcttctttgactccgacaaaggtaaaacccactcttcgggtaaagtgctgtataacgcgcgtatcatcccttaccgtggttcctggctggacttcgaattcgatccgaaggacaacctgttcgtacgtatcgaccgtcgccgtaaactgcctgcgaccatcattctgcgcgccctgaactacaccacagagcagatcctcgacctgttctttgaaaaagttatctttgaaatccgtgataacaagctgcagatggaactggtgccggaacgcctgcgtggtgaaaccgcatcttttgacatcgaagctaacggtaaagtgtacgtagaaaaaggccgccgtatcactgcgcgccacattcgccagctggaaaaagacgacgtcaaactgatcgaagtcccggttgagtacatcgcaggtaaagtggttgctaaagactatattgatgagtctaccggcgagctgatctgcgcagcgaacatggagctgagcctggatctgctggctaagctgagccagtctggtcacaagcgtatcgaaacgctgttcaccaacgatctggatcacggcccatatatctctgaaaccttacgtgtcgacccaactaacgaccgtctgagcgcactggtagaaatctaccgcatgatgcgccctggcgagccgccgactcgtgaagcagctgaaagcctgttcgagaacctgttcttctccgaagaccgttatgacttgtctgcggttggtcgtatgaagttcaaccgttctctgctgcgcgaagaaatcgaaggttccggtatcctgagcaaagacgacatcattgatgttatgaaaaagctcatcgatatccgtaacggtaaaggcgaagtcgatgatatcgaccacctcggcaaccgtcgtatccgttccgttggcgaaatggcggaaaaccagttccgcgttggcctggtacgtgtagagcgtgcggtgaaagagcgtctgtctctgggcgatctggataccctgatgccacaggatatgatcaacgccaagccgatttccgcagcagtgaaagagttcttcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgaatacggcttccttgagactccgtatcgtaaagtgaccgacggtgttgtaactgacgaaattcactacctgtctgctatcgaagaaggcaactacgttatcgcccaggcgaactccaacttggatgaagaaggccacttcgtagaagacctggtaacttgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggccgttccgactctgcgcgctgataagccgctggttggtactggtatggaacgtgctgttgccgttgactccggtgtaactgcggtagctaaacgtggtggtgtcgttcagtacgtggatgcttcccgtatcgttatcaaagttaacgaagacgagatgtatccgggtgaagcaggtatcgacatctacaacctgaccaaatacacccgttctaaccagaacacctgtatcaaccagatgccgtgtgtgtctctgggtgaaccggttgaacgtggcgacgtgctggcagacggtccgtccaccgacctcggtgaactggcgcttggtcagaacatgcgcgtagcgttcatgccgtggaatggttacaacttcgaagactccatcctcgtatccgagcgtgttgttcaggaagaccgtttcaccaccatccacattcaggaactggcgtgtgtgtcccgtgacaccaagctgggtccggaagagatcaccgctgacatcccgaacgtgggtgaagctgcgctctccaaactggatgaatccggtatcgtttacattggtgcggaagtgaccggtggcgacattctggttggtaaggtaacgccgaaaggtgaaactcagctgaccccagaagaaaaactgctgcgtgcgatcttcggtgagaaagcctctgacgttaaagactcttctctgcgcgtaccaaacggtgtatccggtacggttatcgacgttcaggtctttactcgcgatggcgtagaaaaagacaaacgtgcgctggaaatcgaagaaatgcagctcaaacaggcgaagaaagacctgtctgaagaactgcagatcctcgaagcgggtctgttcagccgtatccgtgctgtgctggtagccggtggcgttgaagctgagaagctcgacaaactgccgcgcgatcgctggctggagctgggcctgacagacgaagagaaacaaaatcagctggaacagctggctgagcagtatgacgaactgaaacacgagttcgagaagaaactcgaagcgaaacgccgcaaaatcacccagggcgacgatctggcaccgggcgtgctgaagattgttaaggtatatctggcggttaaacgccgtatccagcctggtgacaagatggcaggtcgtcacggtaacaagggtgtaatttctaagatcaacccgatcgaagatatgccttacgatgaaaacggtacgccggtagacatcgtactgaacccgctgggcgtaccgtctcgtatgaacatcggtcagatcctcgaaacccacctgggtatggctgcgaaaggtatcggcgacaagatcaacgccatgctgaaacagcagcaagaagtcgcgaaactgcgcgaattcatccagcgtgcgtacgatctgggcgctgacgttcgtcagaaagttgacctgagtaccttcagcgatgaagaagttatgcgtctggctgaaaacctgcgcaaaggtatgccaatcgcaacgccggtgttcgacggtgcgaaagaagcagaaattaaagagctgctgaaacttggcgacctgccgacttccggtcagatccgcctgtacgatggtcgcactggtgaacagttcgagcgtccggtaaccgttggttacatgtacatgctgaaactgaaccacctggtcgacgacaagatgcacgcgcgttccaccggttcttacagcctggttactcagcagccgctgggtggtaaggcacagttcggtggtcagcgtttcggggagatggaagtgtgggcgctggaagcatacggcgcagcatacaccctgcaggaaatgctcaccgttaagtctgatgacgtgaacggtcgtaccaagatgtataaaaacatcgtggacggcaaccatcagatggagccgggcatgccagaatccttcaacgtattgttgaaagagattcgttcgctgggtatcaacatcgaactggaagacgagtaattctcgctcaaacaggtcactgctgtcgggttaaaacccgaccgcagcccaagctgggtaccgagctcggtacccgatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaa

SEQ ID NO: 3 - DNA, 3853 bp, Aequorea victoria
ccatcgaatggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatggccagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 4 - DNA, 5027 bp, Aequorea victoria
ggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatggccagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtggtctctgatcacttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatattgcatgctacat

SEQ ID NO: 5 - DNA, 4922 bp, Aequorea victoria
gcatgctacgtagatctagaaataattttgtttaactttaagaaggagatatacaaatggccagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtggtctctgatcacttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatt

SEQ ID NO: 6 - DNA, 42 bp, Aequorea victoria
  misc_feature (12)..(12): n is a, c, g, or t
cacacggtctcnaatggccagcaaaggagaagaacttttcac

SEQ ID NO: 7 - DNA, 33 bp, Aequorea victoria
cacacaagcttacttgtacagctcgtccatgcc

(19) United States
(12) Patent Application Publication
(10) Pub. No.: US 2006/0014197 A1
(43) Pub. Date: Jan. 19, 2006

(54) In vivo screening methods for identifying inhibitors of RNA polymerases

(75) Inventor: Robert C. Landick, Madison, WI (US)

Correspondence Address: GODFREY & KAHN, S.C., 780 N. WATER STREET, MILWAUKEE, WI, 53202, US

(73) Assignee: Wisconsin Alumni Research Foundation

(21) Appl. No.: 11/183,036

(22) Filed: Jul. 15, 2005

Related U.S. Application Data
(60) Provisional application No. 60/588,241, filed on Jul. 15, 2004.

(51) Int. Cl. C12Q 1/68; C12N 15/85

(52) U.S. Cl. 435/6; 435/455

(57) ABSTRACT

In vivo screening methods for identifying inhibitors of RNA polymerase (RNAP) are provided by the present invention. In certain embodiments, methods according to the invention include steps of: (a) expressing a test RNAP in a cell containing a first reporter; (b) expressing a control RNAP in the cell of step (a) that also contains a second different reporter or, alternatively, a different cell that contains the second different reporter, wherein the first and second different reporters distinguish activities of the test and control RNAPs, respectively; (c) contacting the cell or cells of step (b) with a candidate compound; and (d) assaying to obtain a combined signal from the first reporter and second different reporter wherein a unique combined signal identifies the candidate compound as an inhibitor of an RNAP.

### CROSS-REFERENCE TO RELATED APPLICATION

The present application claims the benefit of U.S. Provisional application 60/588,241, filed Jul. 15, 2004, which is incorporated by reference herein in its entirety.

### STATEMENT AS TO RIGHTS TO INVENTIONS MADE UNDER FEDERALLY SPONSORED RESEARCH AND DEVELOPMENT

This work was supported in part by a grant from the National Institutes of Health GM38660. The Government of the United States of America may have certain rights in this invention.

### FIELD OF THE INVENTION

This invention relates generally to methods for identifying novel antibiotics with improved specificity. In particular, the invention is directed to in vivo screening methods for identifying inhibitors of ribonucleic acid polymerases (RNAPs).

### BACKGROUND OF THE INVENTION

Synthesis of RNA from a DNA template is the fundamental step in gene expression. The reaction is catalyzed by an enzyme called RNA polymerase (RNAP). RNAP is the central enzyme in gene expression and, as such, it is the target of a vast array of regulatory signal pathways that control its activity. It also is absolutely essential to all forms of life.

In all cellular organisms (i.e., all organisms except bacteriophage and viruses), RNAP is a multi-subunit enzyme with a generally conserved structure (FIG. 1A). In bacteria, there are generally five subunits: beta′, beta, alpha, alpha and sigma. This basic architecture of multi-subunit RNAPs is conserved throughout the living world, with two large subunits forming the bulk of the enzyme (beta and beta′ in bacteria), a homo- or hetero-dimer of smaller subunits on the periphery of the enzyme involved in assembly (the alpha dimer in bacteria), and at least one accessory subunit (sigma in bacteria). Beta′ and beta are split into two polypeptides in some organisms (Severinov, et al. (1996). J Biol Chem 271, 27969-27974) and can be fused into one polypeptide in others (Zakharova, et al. (1999) J Bacteriol 181, 3857-3859). Together, beta′ and beta form the catalytic core of the enzyme and maintain the nucleic acid scaffold of the transcription elongation complex (TEC; FIGS. 1B & C). Beta′ and beta are homologous to the two largest subunits of eukaryotic RNAPs (RPB1 and RPB2, respectively in yeast RNAP II). Elements of sequence similarity are present in a conserved order in the primary structure of these subunits: A through H in beta′ and A through I in beta′ (Allison, et al. (1985) Cell 42, 599-610: Sweetser, et al. (1987) Proc Natl Acad Sci USA 84, 1192-1196). In the three-dimensional structure of core RNAP, these conserved elements cluster around the active center, with the more divergent regions of the subunits located on the periphery of the enzyme (Zhang, et al. (1999) Cell 98, 811-824).

As shown in FIGS. 1B and C, beta′ and beta form a main channel of the TEC that holds the RNA 3′ OH in the active site, an 8-9 bp RNA:DNA hybrid, duplex DNA in front of the hybrid, and single-stranded RNA upstream from the hybrid. A secondary channel connects the active site to the surrounding solution and may serve as a passageway for entering NTPs, exiting pyrophosphate, or both. Within the main channel, bacterial and eukaryotic RNAPs are nearly identical in structure; thus, the mechanism of transcription by the multisubunit RNAPs of all cellular life forms appears to be the same (Cramer et al. (2001) Science 292, 1863-1876; Zhang et al., 1999; Ederth, et al. (2002). J Biol Chem 277, 37456-37463; Toulokhonov, et al. (2001) Science 292, 730-733.). Nucleotide addition occurs by two-Mg2+-catalyzed SN2 nucleophilic attack of the RNA 3′ OH on the alpha phosphate of a nucleoside triphosphate (NTP); the 3′ nucleotide and the NTP are bound in subsites i and i+1, respectively (Sosunov, et al. (2003). Embo J 22, 2234-2244).

Compounds that inhibit an organism's RNAP are lethal. The best-known inhibitor is derived from the Amanita mushroom, is called alpha-amanitin, and is responsible for about a hundred deaths annually among undiscriminating mushroom hunters. RNAP inhibitors typically are specific for a single class of organisms. Alpha-amanitin, for example, affects higher eukaryotes, but has no effect on bacteria. Conversely, some drugs specifically affect bacterial RNAP. The best known of these is rifampin, which is produced by a fungi and is currently in use as an anti-tuberculosis drug as the rifampin derivative Rifampicin (Rif). Rif is specific for bacterial RNAPs. This specificity of inhibitors occurs for two reasons. First, the inhibitors are often made by one organism to kill another and the producing organism must evolve an inhibitor that is not suicidal. Second, the inhibitors usually bind to the less-conserved parts of the enzyme, where sequence variation can prevent them from working on all RNAPs.

More generally, five inhibitors of bacterial RNAP are well characterized, although many others are known. Rif binds bacterial RNAP in a pocket that contacts nascent RNA in TECs. Rif blocks synthesis of RNAs longer than 2-3 nt, but cannot bind to or inhibit TECs (Campbell, et al. (2001). Cell 104, 901-912, and refs. therein). Alpha-amanitin binds within the secondary channel of eukaryotic RNAPII. Alpha-amanitin inhibits nucleotide addition at all stages of transcription, possibly by restricting movements in the active site of RNAP, partially blocking the secondary channel, or both (Bushnell, et al. (2002) Proc Natl Acad Sci USA 99, 1218-1222, and refs. therein). Many amino-acid substitutions confer resistance to rifampicin or alpha-amanitin; importantly, all occur in the inhibitor-binding sites (Bushnell et al., 2002; Campbell et al., 2001, and references therein). Inhibitors of bacterial RNAPs of class CBR703 were recently described (Artsimovitch, et al. (2003). Science 302, 650-654). CBR703 inhibitors block nucleotide addition allosterically by binding to an outside surface of RNAP. Two other inhibitors, streptolydigin and microcin J25, which affect bacterial RNAP similarly to alpha-amanitin's effect on RNAPII, bind near the secondary channel and active site (Severinov, et al. (1995) J Biol Chem 270, 23926-23929; Yuzenkova, et al. (2002) J Biol Chem 277, 50867-50875, and refs. therein).

The general need for new antibiotics has been widely publicized and need not be reiterated here. Suffice it to say that existing antibiotics, to which much of the improvement in human health over the course of the last century can be attributed, have a limited useful lifetime. Over time, bacteria acquire resistance against antibiotics to which they are exposed. The available arsenal against some of the most deadly bacterial pathogens is now nearing depletion (Shales, et al. (2004) ASM News 70, 275-281; Bush, K. (2004) ASM News 70, 282-287; Shea, K. M. (2003). Pediatrics 112, 253-258; Waugh, et al. (2002) Sci Prog 85, 73-88.). The current concern that some bacterial species (e.g., Bacillus anthracis) may become bioweapons in the hands of terrorists only heightens the pressing need for new antibiotics. It also highlights the desirability of finding inhibitors that optimally target particular bacterial species. Because RNAP is essential to growth of all bacteria, it is an extremely valuable drug target.

The prospects of obtaining species-specific RNAP inhibitors is especially attractive because it could yield drugs targeted to pathogenic bacteria that are less lethal to the normal bacterial flora present in healthy individuals. In particular, methods to identify new bacterial RNAP inhibitors that function within a bacterium (i.e., enter the bacterial cell and are not inactivated within the cell), and affect RNAPs from one class of bacteria (e.g., pathogenic gram-positive bacteria) while not affecting RNAPs from another class of bacteria (e.g., gram-negative bacteria) would fulfill a long felt need in the vitally important search for new and more specific antibiotics.

### SUMMARY OF THE INVENTION

The present invention is directed to in vivo screening methods for identifying inhibitors of an RNAP which function within an organism such as a bacterium or a human cell. The invention has a specific adaptation that allows identification of inhibitors that affect certain RNAPs while not affecting other RNAPs.

Accordingly, the invention provides an in vivo screening method for identifying an inhibitor of a ribonucleic acid polymerase (RNAP) which includes steps of: (a) expressing a test RNAP in a cell containing a first reporter; (b) expressing a control RNAP in the cell of step (a) that also contains a second different reporter or, alternatively, a different cell that contains the second different reporter, wherein the first and second different reporters distinguish activities of the test and control RNAPs, respectively; (c) contacting the cell or cells of step (b) with a candidate compound; and (d) assaying to obtain a combined signal from the first reporter and second different reporter wherein a unique combined signal identifies the candidate compound as an inhibitor of an RNAP. In some embodiments, a combined signal comprises two or more homologous signals, e.g. two different fluorescent wavelengths. In some preferred embodiments, a combined signal is read as a single signal, e.g. red and green fluorescence emissions that combined appear as yellow. In other embodiments, a combined signal is read as the sum of two or more discrete heterologous signals, e.g. a chemiluminescent signal and a fluorescent signal.

In certain embodiments, the inhibitor identified by the method is a specific inhibitor of the test RNAP and selective inhibition of the test RNAP relative to the control RNAP is indicated by the unique combined signal. In a similar fashion, the present invention also facilitates the identification of general RNAP inhibitors wherein general inhibition is-indicated by the combined signal from the first reporter and second different reporter.

According to certain embodiments of the invention, activities of the test and control RNAPs are respectively distinguished by use of reporters selectably-transcribed by the test and control RNAPs. Such selectable transcription allows the test and control RNAPs to be distinguished not only from each other but from the activity of host cell RNAP(s). Alternatively, the test and control RNAPs are drug resistant and their activities are distinguished from activities of a host RNAP also contained within the cell or cells by the addition of a drug which inhibits said host RNAP but not the drug resistant test and control RNAPs. Drug resistant RNAPs may be resistant to, for example, α-amanitin, streptolydigin, or rifampicin. Various drug resistant RNAP variants are known in the field. In other embodiments, the host RNAP is inhibited through the use of strains with temperature sensitive mutations that inactivate the host RNAP. Accordingly, a temperature shift allows inhibition of host RNAP prior to induction of reporters.

In certain embodiments, the invention provides an in vivo screening method for identifying an inhibitor of a ribonucleic acid polymerase in which test and control RNAPs are contained within separate cells, respectively. In certain other embodiments, the invention provides an in vivo screening method for identifying an inhibitor of a ribonucleic acid polymerase (RNAP) in which test and control RNAPs are contained within a single cell. Despite such procedural differences, methods described and claimed herein are fundamentally similar in terms of assaying a combined signal from a combination of reporter constructs, each reporter providing a measure of a particular RNAP's activity.

Methods according to the invention are extremely robust in terms of, among other factors, assayable putative inhibitors (also termed “candidate compounds”), cells and reporters. In some embodiments, the putative inhibitors are provided as reagents in the reaction or growth medium. In other embodiments, the putative inhibitors are encoded in DNA, e.g. plasmids, cosmids, plastids, and subjected to expression in the presence of the cell or cells.

Cell types useful in the invention include both bacterial and eukaryotic cell types. E. coli is a particularly-preferred bacterial cell type useful in the invention.

Although various reporter systems may be adapted by only routine modification for use in the present invention, preferred embodiments utilize differently-colored fluorescent proteins (e.g., mutant variants of green fluorescent protein (GFP)) in the reporter roles. In other embodiments, the reporter may be a chemiluminescent protein such as luciferase or any other reporter whose expression can be dependent on the activity of an RNAP.

The present invention offers methods differing from those currently available because screening for inhibitors can be performed in living cells (e.g., a bacterium such as E. coli), and because it is possible to identify inhibitors of RNAP from one class of organism (e.g., gram-positive pathogens) that do not affect the RNAP in another class of organism (e.g., gram-negative bacteria). Additionally, the methods of the invention can be used to distinguish inhibitors of any bacterial, bacteriophage or eukaryotic RNAPs, not just that of E. coli, and are advantageous because they will only identify inhibitors that will enter the cell and that will work within the bacterial cytoplasm or within a eukaryotic cell. The methods can therefore be used to efficiently identify lead compounds for development of either broad-spectrum or narrow-spectrum antibiotics. The methods are especially efficient at identifying lead compounds for narrow spectrum antibiotics that will act on specific classes of pathogenic bacteria without killing the entire bacterial flora in a host.

Other objects, features and advantages of the present invention will become apparent after review of the specification, claims and drawings.

### BRIEF DESCRIPTION OF THE DRAWINGS

FIG. 1. Structure of RNAP and TECs. A. The structure of bacterial RNAP. The alpha subunits are located primarily on the backside of the enzyme. In the front view shown, the two large subunits, beta and beta′, form the active-site cleft, which is centered around the catalytic Mg2+ ion. In this view, the location of the binding sites for Rif and alpha-amanitin are located to the left and right, respectively, of the Mg2+ ion (note that alpha-amanitin binds eukaryotic RNAPII, not bacterial RNAP, but is shown here for illustrative purposes). B. A diagram of the structure of the TEC. Only the beta and beta′ subunits are shown. The view is similar to that shown in A and C, but rotated upwards ˜30°. C. A TEC structure shown in the same orientation as A. A domain of beta′ called the clamp rotates down over the RNA:DNA hybrid. Alpha-amanitin, but not Rif, can bind to and inhibit the TEC.

FIG. 2. A. Sequence of TetR-regulated promoter for expression of GFP. The black boxes are promoter elements. The underlined sequences are the TetR binding sites, which are symmetrical around the black diamonds. Arrow indicates the transcription start. B. Cassette for integration into E. coli chromosome.

FIG. 3 illustrates an RNAP overexpression plasmid useful in carrying out the present invention.

FIG. 4 depicts an experimental scheme, further explained in the Examples section.

FIG. 5 illustrates an approach to create overexpression plasmids for bacterial RNAPs. A. Each gene is separately amplified and validated by sequencing before ligating into the overexpression plasmid. B. The validated genes are transferred to pRNAPexpress by ligation between sites that are unique in the plasmid.

FIG. 6 depicts a schematic representation of a preferred in vivo method useful to identify specific inhibitors of RNAPs according to the present invention.

FIG. 7 Expression of α-amanitin resistant RNAP. FIG. 7A depicts a two plasmid expression and reporter system for α-amanatin resistant RNAP. FIG. 7B presents a graph showing the effects of adding α-amanatin and/or doxycycline to cells carrying such a two-plasmid system.

FIG. 8 depicts a plasmid system for screening for inhibitors of a bacterial or bacteriophage RNAP in a eukaryotic cell. FIG. 8A shows a schematic illustration of a multi-plasmid system comprising a plasmid encoding a bacterial RNAP, a plasmid comprising a reporter gene indicative of transcription by the bacterial RNAP, and a plasmid comprising a reporter gene indicative of transcription by the eukaryotic host RNAP. FIG. 8B presents an example of the kind of reporter gene expression data obtained using such a multi-plasmid system.

FIG. 9 Demonstration of the GFP reporter assay. A. Diagram of the cell in which the assay is performed. pβ is either pRM546 or pRM547, which encode IPTG-inducible wild-type E. coli rpoB or mutant rpoB(S531F; Rif^{r}). The reporter plasmid is pAI8 as described in the test. B. GFP fluorescence measured after various treatments of cultures containing pRM546 (wild-type β) or pRM547 (Rif^{r} β). The results are the averages of three independent cultures, with standard deviations in results indicated by the error bars. The diagram on the left illustrates the order of treatment of the cultures. See text for details of culture growth and treatment.

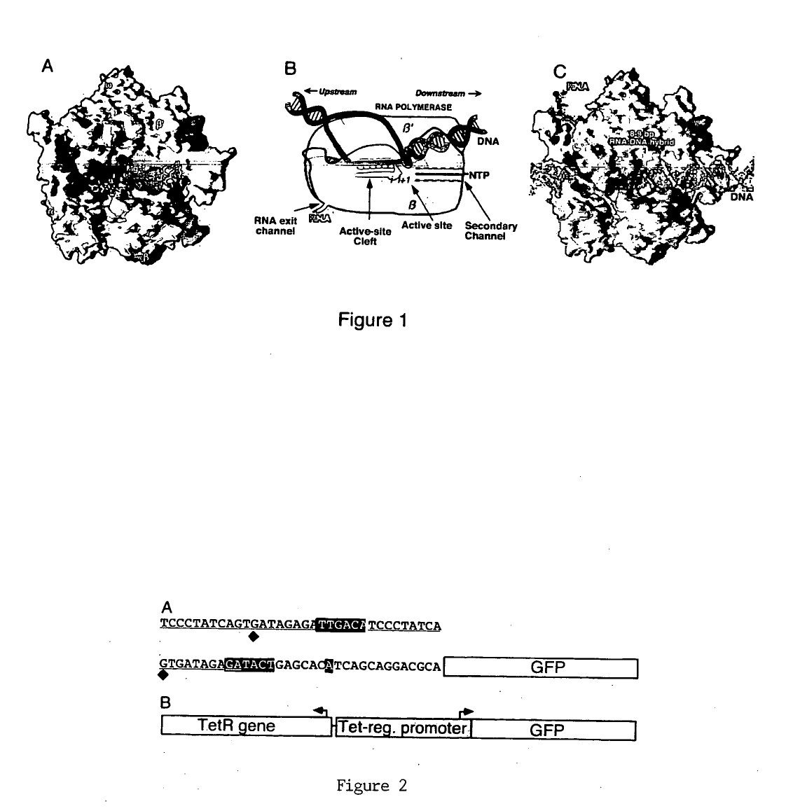

### DETAILED DESCRIPTION OF THE INVENTION

### I. IN GENERAL

Before the present methods are described, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the polypeptides, polynucleotides, cell lines, vectors, and methodologies which are reported in the publications which might be used in connection with the invention. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

### II. THE INVENTION

The present invention provides methods useful in the identification of inhibitors of RNAP in living organisms, while simultaneously facilitating the identification of inhibitors for a particular RNAP of interest. Certain methods according to the invention include the steps of: (1) expressing in a cell a test RNAP either in a form resistant to an antibiotic, preferably rifampicin, or specific for a reporter, preferably a fluorescent or chemiluminescent protein; (2) when necessary, inhibiting the host RNAP of the bacteria with an antibiotic; (3) contacting the bacteria with a candidate compound and (4) allowing expression of a reporter (e.g., by de-repression of a promoter associated with a fluorescent or chemiluminescent protein); and (5) screening for inhibition of the test RNAP by failure of fluorescence or chemiluminescence to be generated from the reporter.

Methods according to the invention are made specific for the test RNAP of interest by including in the assay a different RNAP, termed a control RNAP, and a second different reporter, preferably, a differently-colored fluorescent or chemiluminescent protein which provides a signal commensurate with the control RNAP's activity. The control RNAP and second different reporter are either contained within the same cell as the test RNAP and first reporter or, alternatively, provided in a different cell. In the first approach, the first and second different reporters are selectably-transcribed by the test and control RNAP, respectively, thereby allowing the method to distinguish between activities of test versus control RNAPs in a single cell source. In the methods described herein, if only the test RNAP, and not the control RNAP, is inhibited, then a unique combined reporter signal distinguishable from other reporter signals (e.g., signals generated in response to nonspecific inhibitors) will result and be detected in a multicolor analysis.

Because the RNAPs in question can be virtually any RNAP that can be expressed in functional form in cells like E. coli or human cells, the method can be used to find inhibitors selective for virtually any arbitrarily defined class of RNAP. Variations of this assay include, but are not limited to, (i) inhibition of a xenogeneic RNAP expressed in an E. coli strain (ii) selective inhibition of only one of two different xenogeneic RNAPs expressed in the two E. coli strains; (iii) selective inhibition of an RNAP containing a particular domain or site compared to a second RNAP lacking that domain or site; (iv) inhibition of a bacterial RNAP compared to a phage or viral RNAP, such as T7 RNA polymerase; (v) inhibition of a phage or viral RNA polymerase, such as T7 RNA polymerase, compared to a bacterial RNAP; (vi) inhibition of a viral or bacterial RNAP expressed in a human cell; (vii) selective inhibition of only one of two different xenogeneic RNAPs expressed in the two human cell lines; and (viii) expression of a eukaryotic RNAP subunit that can combine with host-produced subunits in a cell such as a human cell line and yield and RNAP that can remain active when the host RNAP is inhibited by a compound such as amanitin.

In methods according to the invention, an initial step calls for the selection of appropriate bacteria and the expression in that bacteria of a test RNAP. Suitable bacteria for use in the invention include, but are not limited to, Escherichia, Salmonella, Serratia, Proteus, Aerobacter, and Bacillus, with E. coli being the preferred host bacteria. Bacterial strain selection may be based on the presence of a host RNAP having sensitivity to a particular antibiotic, as called for by various embodiments of the present invention. As used herein, a “test RNAP” shall refer to an RNAP against which a specific inhibitor is desired. Test RNAPs can be virtually any RNAP that can be expressed in the selected bacteria. The present invention is particularly well suited to identify inhibitors of bacterial RNAPs in bacteria or human cells and of eukaryotic RPB1 subunits able to combine with human subunits in human cells to yield a functional RNAPII.

As noted above, in some embodiments specificity can be achieved in the present invention by the inclusion of a second cell source grown separately or in combined culture with the first cell source and expressing a control RNAP. As used herein, the term “control RNAP” shall mean an RNAP against which the test RNAP is being compared, with the intent to identify inhibitors specific for the test RNAP but preferably having no significant inhibitory effect on the control RNAP. As such, the methods can be used to find inhibitors selective for virtually any arbitrarily defined class of RNAP. Of course, inhibitors which inhibit both test and control RNAPs may also be identified by the present invention and therefore the invention, in another aspect, further provides an approach for identifying general RNAP inhibitors, as well as specific inhibitors.

Transformation of nucleic acids encoding the test and control RNAPs into the host bacteria may be achieved by various techniques well known in the art for creating recombinant bacteria. See, for example, methods in Molecular Cloning A Laboratory Manual, 2nd Ed., ed. by Sambrook, Fritsch and Maniatis (Cold Spring Harbor Laboratory Press: 1989). Transformation of nucleic acids encoding the test and control RNAPs into the host mammalian cells may be achieved by various techniques well known in the art for transfecting mammalian cells or creating recombinant mammalian cell lines. See, for example, DNA Transfer to Cultured Cells (K Ravid and R I Freshney Eds) John Wiley and Sons, New York, 1998.

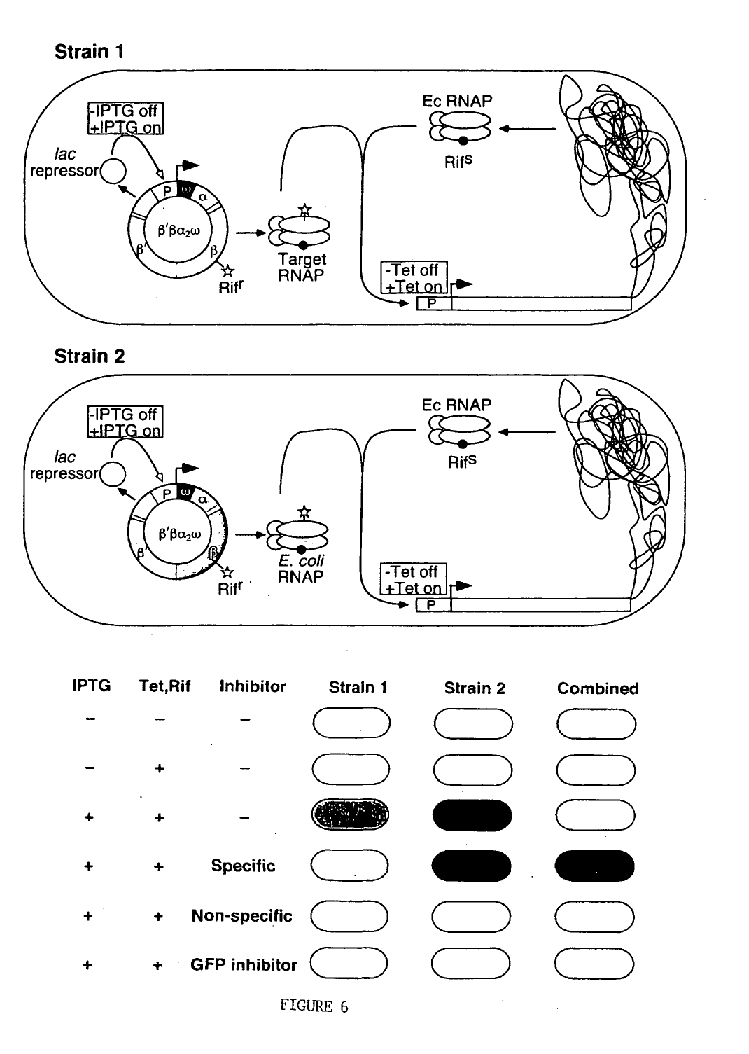

Reference is now made to FIG. 6 which depicts a preferred in vivo method for identifying a specific inhibitor of an RNAP. Expression of test and control RNAPs is preferably independently regulated by inducible promoters, most preferably expression constructs under the control of lacR, the repression of which may be relieved by the addition to the culture of, for example, isopropyl-beta-D-thiogalactopyranoside (IPTG). Expression constructs encoding beta, beta′, alpha and sigma subunits under lacR control are shown schematically in FIG. 6 and are further detailed in the Example section. As alternatives to the lacR system, inducible promoters for expression of RNAPs as called for in the invention also include but are not limited to the AraC-araBAD promoter system, the RhaR-rhaB promoter system, the RhaS-rhaS promoter system, an iron-inducible promoter controlled by the Fur protein, and the T7 RNAP promoter in cells that express T7 RNAP from an inducible source (e.g., such as the BL21lambdaDE3 system. The araC-araBAD promoter system is described in Guzman, et al. (1995) J Bacteriol 177, 4121-4130. The RhaR-rhaB promoter system and RhaS-rhas promoter system is described in Haldimann and Wanner. (2001) J. Bact 183, 6384-6393. Regulation by the Fur repressor is described in de Lorenzo et al. (1988) J Mol Biol 203, 875-884. The BL21lambdaDE3 system is described in Studier et al. (1990) Methods Enzymol 185, 60-89.

In mammalian cells, the test RNAP or test RNAP subunit may be expressed from a variety of suitable promoters that include but are not limited to the Adenovirus major late promoter, the CMV promoter, the HIV-1 promoter, or the DHFR promoter. Conditional expression in mammalian cells can be achieved be use of a regulated promoters that include but are not limited to the Tet-ON or Tet-OFF CMV promoter derivatives sold by Invitrogen.

The various RNAP subunits may be encoded by a single construct, as illustrated, or may be encoded by multiple constructs; e.g., the beta and beta′ subunits may be encoded by a nucleic acid present in a plasmid separate from a plasmid carrying sequences encoding the remaining sigma and alpha subunits. In certain embodiments, the beta and beta′ subunits are fused so that hybrid combinations may not arise with the homologous subunits of the host's RNAP. In other embodiments, only one subunit of an RNAP may be expressed and allowed to combine with host subunits to produce a functional RNAP.

In some preferred embodiments, a reporter plasmid is included which comprises one or more genes under the control of a promoter recognized by the test and control RNAPs but not by the host RNAP. For example, bacterial RNAPs expressed in eukaryotic host cells will transcribe reporter genes under the control of bacterial promoters while the host eukaryotic RNAP will not. In some embodiments, an alternative sigma is used to control expression of reporter genes Examples of such RNAP-promoter systems suitable for testing bacterial RNAPs expressed in bacterial cells include alternative sigma factors and their associated promoters, e.g. σ³² and σ⁵⁴ in E. coli and σ^{B} in Bacillus subtilis. In some embodiments, the host RNAP will not transcribe genes under the control of such promoters while the plasmid-encoded test and control RNAPs will.

In other preferred embodiments, the combined cultures of cells expressing the test RNAP and the control RNAP, a pre-selected antibiotic is added to inhibit the host RNAP expressed by the host cells when needed. Transcription by host RNAP is shut down and the bacterial cell thusly becomes reliant on the test or control RNAP for transcriptional activity. Alternatively, the reporter may be expressed from a promoter that only is recognized by the test RNAP. Antibiotics useful in the present invention inhibit the activity of naturally-occurring RNAPs but against which at least one resistance-conferring mutation is known and applicable in the test and control RNAP. Such antibiotics include, but are not limited to, rifampicin, streptolydigin, microcin J25, lipiarmycin, sorangicin, myxopyronins, or the CBR703 class of inhibitors, of which rifampicin is the most preferred for prokaryotic RNAP. For eukaryotic RNAP, alpha-amanatin is preferred (Bushnell 2002). When necessary, genetically altered cells, such as tolC mutants, or chemical treatment such as incubation with Na₂EDTA can be used to promote uptake or prevent efflux of the antibiotics. Specific mutations bestowing rifampicin resistance on the RNAPs useful in the invention are described in Campbell et al., 2001, in Garibyan, et al. (2003). DNA Repair (Amst) 2, 593-608, and in references cited therein (e.g., Jin & Gross (1989) J Bacteriol 171:5229; Singer et al. (1993) J Mol Biol 231:1; Jin et al. (1988) J Mol Biol 204:247; Jin et al. (1988) J Mol Biol 202:245; Jin & Gross (1988) J Mol Biol 202:45; and Ramaswamy & Musser (1998) Tuber Lung Dis 79:3)

Particularly preferred rifampicin mutations are Ser531 to Phe or Asp514 to Val (E. coli numbering) mutations in the beta subunits which are strong rifampicin resistance (Rif-r) mutants that minimally perturb RNAP activity. It should be noted that some bacterial RNAPs may exhibit natural Rif-resistance (eg, see T. aquaticus RNAP as described in Campbell et al.). Such RNAPs possessing natural Rif-resistance are certainly envisioned as useful in the present invention and, as one of skill will appreciate, actually simplify carrying out methods according to the invention by doing away with the step of selecting a suitable antibiotic resistant RNAP. Alternatively, and as also described herein, the expression of the reporter may depend solely on the test RNAP and the use of the inhibitory antibiotic may be unnecessary in certain alternative embodiments of the invention.

Rifampicin, which binds firmly to the beta subunit of bacterial RNAP, completely blocks productive initiation of RNA chains by the polymerase in vitro and in vivo. The polymerase-rifampicin complex apparently fails in performing the translocation step that follows formation of the first or second phosphodiester bond. The inactive enzyme complex, bound at the promoter site, becomes an effective barrier to transcription through this region by an active RNAP molecule. Bacterial cells gain resistance to rifampicin by virtue of an altered beta subunit that fails to bind the drug. Numerous rifampicin-resistant mutant RNAPs show altered transcription properties. Mutations to rifampicin resistance map in three separate regions within a 200 amino acid stretch in the center of the 1342 residue beta subunit and define a domain termed the rifampicin-binding pocket. Jin D. J., Gross C. A. (1998) J. Mol. Biol. 202:45.

In other embodiments, the use of an antibiotic to inhibit the cellular RNA polymerase is avoided through use of strains with temperature sensitive mutations that inactivate the cellular RNA polymerase. For instance, a mutation in the beta′ subunit of E. coli RNA polymerase that changes Gly1360 to Asp (rpoC1) blocks RNA polymerase activity at 42° C., as does the rpoC397 mutation that replaces 52 amino acids distal to position 1355 with 23 unnatural residues (Gross, et al. (1976) Mol. Gen. Genet. 147:337-341; Gross, et al. (1977) Eur. J. Biochem. 81:333-338; Christie, et al. (1996) J. Bacteriol. 178:6991-6993; Nedea, et al. (1999) J. Bacteriol. 181:2663-2665). Thus, instead of adding, for example, rifampicin to inhibit the endogenous RNA polymerase, the use of strains bearing temperature-sensitive RNA polymerase mutations like rpoC1 and rpoC397 that block RNA polymerase activity after a temperature shift allow inhibition of endogenous RNA polymerase by temperature shift prior to the induction of reporter constructs.

Such methods can be generalized to any cellular RNA polymerase, eukaryotic, archaeal, or prokaryotic, because a mutation in eukaryotic RNA polymerase II in Saccharomyces cerevisiae at the position homologous to the G1360D rpoC1 mutation (called rpb1-1 in Saccharomyces cerevisiae RNA polymerase II and causing the G1437D substitution in the RPB1 subunit; Scafe, et al. (1990) Mol. Cell. Biol. 10: 1270-1275) exhibits the same property of inactivating cellular RNA polymerase II upon temperature shift. Since RNA polymerase II is evolutionarily distant from E. coli RNA polymerase, the ability to produce temperature sensitive enzyme activity by a substitution at this position should be a universal property of multisubunit RNA polymerases in all bacteria, archaea, and eukaryotes that contain this conserved residue. For the purpose of these methods, any mutation that confers temperature-sensitive activity on RNA polymerase or any other condition-dependency on RNA polymerase activity will be applicable to inhibit an endogenous RNA polymerase and allow assay of a foreign RNA polymerase by subsequent induction of a reporter.

Referring again to FIG. 6, expression of reporter proteins, most preferably fluorescent proteins (e.g., GFPs) or chemiluminescent proteins (e.g, luciferases), is subsequently induced in the cells of the combined culture. Such reporter proteins are encoded by nucleic acids in recombinant vectors or are integrated into the bacterial genome. Control of the nucleic acid expression is preferably by an inducible promoter. Preparation of suitable reporter constructs may be carried out using standard recombinant methodology. In a preferred embodiment, the inducible promoter is the inducible tetR system. Other suitable inducible promoters include, but not limited to, the AraC-araBAD promoter system, the RhaR-rhaB promoter system, RhaS-Rhar, an iron-inducible promoter controlled by the Fur protein, and the T7 RNAP promoter in cells that express T7 RNAP from an inducible source (e.g., such as the BL21lambdaDE3 system. In mammalian cells, the reporter may be expressed from any suitable bacteriophage RNAP or bacterial RNAP promoter because these promoters will not be recognized by the host RNAPs. In the case where mammalian test RNAP subunits are used, the reporter may be expressed from regulated promoters that include but are not limited to the Tet-ON or Tet-OFF CMV promoter derivatives sold by Invitrogen. The tetR system is described in Lutz & Bujard (1997) Nucleic Acids Res 25:1203. The araC-araBAD promoter system is described in Guzman, et al. (1995). J Bacteriol 177, 4121-4130. The RhaR-rhaB promoter system and RhaS-rhas promoter system is described in Haldimann and Wanner. (2001) J. Bact 183, 6384-6393. Regulation by the Fur repressor is described in de Lorenzo et al. (1988) J Mol Biol 203, 875-884. The BL21lambdaDE3 system is described in Studier et al. (1990). Methods Enzymol 185, 60-89.

It is assumed in various embodiments of the invention that a xenogeneic RNAP will function with the E. coli sigma70 factor using a near-consensus promoter sequence. There is precedence for this assumption in that B. subtilis RNAP is known to use E. coli sigma70 in vitro (Artsimovitch et al. (2000) J. Bacteriol. 182:6027). However, there are some reports that T. aquaticus RNAP cannot act equivalently (Minakhin et al. (2001) J Bacteriol 183:71). Thus, for bacteria very distantly related to E. coli, it may be necessary to express the homologous sigma factor (e.g., T aquaticus sigmaA in the case of T. aquaticus). The xenogeneic sigma may be included in the RNAP overexpression plasmid or it may be expressed from a separate, compatible plasmid. It may also be fused to the C-terminus of the bacterial RNAP beta′ subunit, where it has been shown to function normally (Mooney et al., 2003). In any case, the xenogeneic sigma would most likely recognize the promoter for the reporter, as this has proven to work for most foreign sigmas (eg, Minakhin et al. (2001) J Bacteriol 183:71, Jaurin & Cohen (1984) Gene 28:83). In the case that it does not, the reporter promoter sequence could be altered to allow transcription of the reporter by the complex of xenogeneic RNAP and xenogeneic sigma. In this case, expression of the reporter may become specific for test and control RNAPs and not expressed by the host RNAP, as will definitely be the case when bacterial RNAPs or bacteriopahge RNAPs are tested in mammalian cells. In such cases, the use of antibiotics to inhibit the host RNAP will be unnecessary.

The reporter proteins utilized in the present invention are preferably fluorescent proteins including, but not limited to, differently-colored green fluorescent proteins (GFPs) such as the available mutant variants of the Aequorea victoria gene. A wide assortment of fluorescent proteins are currently available beyond the A. victoria-related options for carrying out multicolor fluorescent analysis including, for example, the reef coral fluorescent proteins (AmCyan, ZsGreen, ZsYellow, AsRed2, DsRed2, and HcRed1), available from BD Biosciences Clontech. Suitable inducible expression constructs encoding fluorescent proteins useful in the present invention may be constructed by one of skill in the art following review of the present disclosure. References describing exemplary reporter proteins include: Bevis et al. (2002). Nat. Biotechnol. 20:83-87; Gurskaya, et al. (2001) FEBS Lett. 507:16-20; Lukyanov, et al. (2000) J. Biol. Chem. 275:25879-25882; and Matz, et al. (1999) Nat. Biotechnol. 17:969-973.

Alternatively, the reporter proteins can be chemiluminescent proteins such as luciferase or any other protein that can be made to generate light or some other recognizable signal and whose expression can be made dependent on the test and control RNAPs.

Prior to inducing expression of the respective fluorescent reporters, the cells of the combined culture are contacted with a candidate compound. The candidate compound is a potential RNAP inhibitor which displays specificity to the test RNAP over the control RNAP. Candidate compounds include, but are not limited to, 1) peptides such as soluble peptides; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The candidate compounds may be obtained using any of the numerous approaches in combinatorial library methods known in the art, including: biological libraries; spatially addressable parallel solid phase or solution phase libraries; synthetic library methods requiring deconvolution; the one-bead one-compound library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example, in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed, Engl. 33:2061; and Gallop et al. (1994) J. Med. Chem. 37:1233.

Libraries of compounds may be presented in various formats including, for example, solution (e.g., Houghten (1992) Biotechniques 13:412-421), plasmids (Cull et al. (1992) Proc. Natl. Acad. Sci. USA 89:1865-1869), BAC libraries (WO0181567A2) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

As shown in FIG. 6, upon addition of the candidate compound to the combined culture, the combined fluorescence signal from the first and second reporters is then assayed. In general, this assay is carried out by multiwell detectors such as multiwell fluorimeters, multiwell luminometers, or other multiwell detectors of signals such as light, radioactivity, or that are now in common use for automated screening of libraries of candidate inhibitor compounds. In the preferred embodiment using two differently colored fluorescent proteins, the multiwell detector will be capable of discriminating the two different color signals by use of either multiple channel detectors with different wavelength specificities (e.g., achieved by different filters or wavelength-tunable detector) or with changeable detector-specificities that can sample the same well at different wavelengths in sequential readings. Exemplary instruments suitable for such detection include the CYTOFLUOR 4000 Multiwell Plate Reader (Applied Biosystems, Inc., Foster City, Calif.) and the GENios PRO (Tecan, Zurich, Switzerland). The assay can also be carried out by automated flow cytometry in which the cell samples are flowed past a detector that can distinguish the fluorescent or chemiluminescent signal from individual cells, quantitating both the signal intensity and the number of cells with each characteristic signal intensity. Exemplary instruments include the CYFLO system (Partec, Munster, Germany), the FACSVantage SE and the FACSCalibur (BD Biosciences-Immunocytometry Systems, San Jose, Calif.), and EPICS Altra Systems (Beckman Coulter, Fullerton, Calif.).

Selective inhibition of the test RNAP by the candidate compound is indicated by a unique combined fluorescent signal as compared to: (i) a combined fluorescent signal generated when both test and control RNAPs are nonspecifically inhibited; and (ii) the combined fluorescent signal generated in the absence of the candidate compound. The unique combined fluorescent signal therefore identifies the candidate compound as a specific inhibitor of the test RNAP. Exemplary controls for assays according to the invention are depicted in FIG. 6.

Some inhibitors of the reporter enzymes themselves or of steps in the production of the reporter other than transcription (such as translation) may be detected by some versions of the screening protocol. Such inhibitors can be identified either using in vitro assays of the reporter enzymes or by expressing the reporter with a heterologous RNAP such as T7 RNAP that will not be affected a specific inhibitor (in the case of inhibitors of translation or of the reporter, inhibition also will be observed with T7 RNAP based expression).

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### III. EXAMPLES

### Example 1

### Demonstration of Conditional Expression of a Reporter Gene by E. coli RNAP Over Expressed from a Plasmid

Reporter gene constructs in which expression of a reporter protein is under control of the Tet repressor may be constructed as follows. The reporter protein (at least for the purposes of this example) will be the green fluorescent protein (GFP) from the jellyfish A. Victoria, a readily available research tool. Several properties of Tet repressor make it the preferred expression system. It is a simple bacterial repressor that tightly regulates promoters when its operator site is located within a promoter sequence. It can be readily supplied to bacteria without unwanted side-effects by expression from a copy of the tet repressor gene integrated in the bacterial chromosome. Finally, and most importantly, it regulates transcription initiation at a promoter in a simple manner, such that addition of an inducer will allow transcription to occur without the need for additional protein synthesis.

As illustrated in FIG. 2, a TetR-controlled promoter sequence may be inserted upstream from a selected GFP gene. The promoter DNA may be synthesized using oligonucleotides ligated upstream from a promoter-less GFP gene in a plasmid that contains the TetR gene transcribed in the opposite direction. This entire cassette may then be transferred to the chromosome of E. coli by recombination, resulting in a stable integrant that can be readily moved from strain to strain by phage P1 transduction.

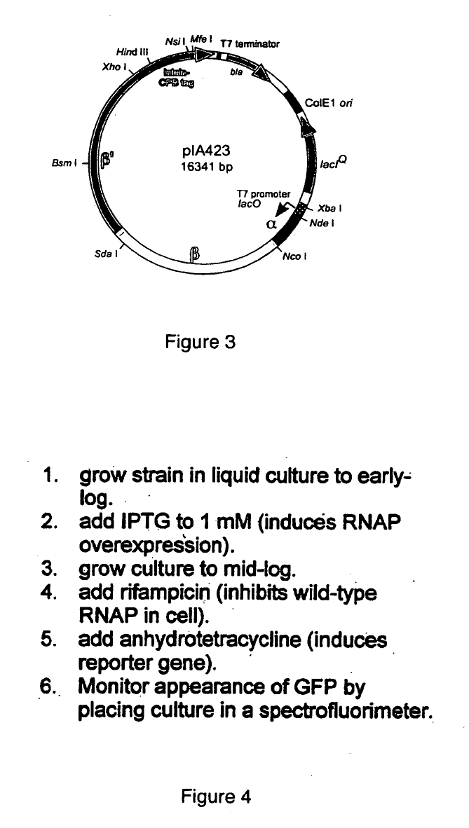

To obtain an RNAP overexpression plasmid suitable for use in the invention, a previously-described plasmid, pIA423 (Artsimovitch, et al. (2003) J Biol Chem 278, 12344-12355, FIG. 3) may be utilized. pIA423 expresses E. coli RNAP under control of the lac repressor (LacR; encoded by lacIQ). To obtain a plasmid that expresses a version of RNAP that can be selected by addition of rifampicin (Rif) to cultures, a mutation encoding the Rif-resistance mutation rpoB(S531F) may be introduced into the rpoB gene of pIA423. This manipulation can be accomplished readily by standard techniques (e.g., Landick, et al. (1990) J Bacteriol 172, 2844-2854.). The resulting plasmid will then express a Rif-resistant RNAP only when induced by addition of IPTG to bacterial cultures.

The Rif^{r}-RNAP overexpression plasmid may then be combined with the GFP-reporter strain and tested for specific expression of the reporter by the plasmid-encoded RNAP. Construction of this test strain is according to transformation techniques known in the art. Steps depicted in FIG. 4 may then be carried out using the thusly-constructed strain. First, the strain is grown in liquid culture to early log phase. Expression of Rif^{r}-RNAP is then induced by adding IPTG. After continuing growth long enough to let the RNAP accumulate, Rif is then added to the culture to inhibit the wild-type RNAP present in the cell. Anhydrotetracycline is then added to remove TetR from the promoter in front of the GFP reporter gene in the cell. Since the wild-type cellular RNAP will be inhibited, only the RNAP made from the overexpression plasmid, which is resistant to Rif, will be able to make mRNA from the reporter gene. The appearance of GFP in the culture is then monitored using a spectrofluorimeter at the emission wavelength for the respective GFP reporter.

Two potential complications in the above-described scheme may be avoided as follows. First, the culture medium, Rif, or anhydrotetracycline may interfere with detection of GFP fluorescence. If this happens, the cells may be washed free of culture medium and the added compounds prior to measuring fluorescence. Alternatively, the GFP fluorescence may be measured on a single-cell level using a fluorescence microscope. A second complication may arise if Rif fails to inhibit the cellular RNAP. This occurrence may be controlled for by using a Rif-sensitive version of the RNAP overexpression plasmid. In this case, Rif should completely eliminate the appearance of GFP in the experimental procedure set forth in FIG. 4.

### Example 2

### Overexpression in E. coli of a Target RNAP from a Pathogenic Bacterium

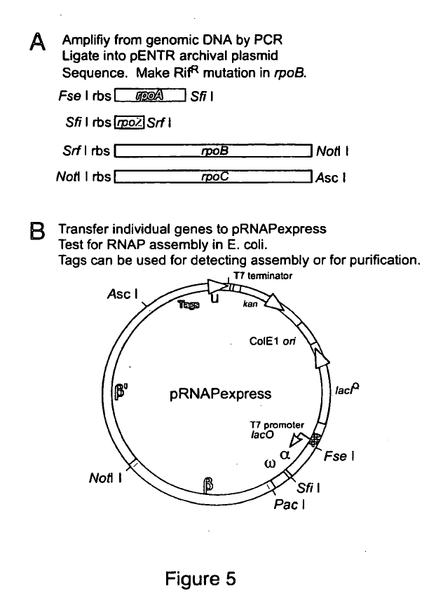

This example describes the assembly of overexpression plasmids for bacterial RNAPs other than those of E. coli. Two attractive pathogenic bacteria for this manipulation are Bacillus anthracis and Streptococcus pneumoniae. Genomic DNAs for B. anthracis and S. pneumoniae are available from, for example, the American Type Tissue Collection. Primers may be designed to facilitate amplifying the genes for the four subunits of RNAP from each bacterium (rpoA encoding alpha, rpoB encoding beta, rpoC encoding beta′, and rpoZ encoding sigma) such that each gene carries unique restriction endonuclease recognition sites at its ends and a ribosome-binding site (rbs) at its 5′ end (see FIGS. 5A and B; the restriction enzymes chosen have 8-bp recognition sequences and do not appear in the bacterial RNAP subunits or the pRNAPexpress backbone). Initially, the genes may be ligated or recombined into the archival plasmids pCR-Blunt (Invitrogen), pCR-BluntIITOPO (Invitrogen), pDONR (Invitrogen), pENTR (Invitrogen), or pCR-Script (Stratagene) using PCR-product cloning methodology. This will allow the capture of the rpo genes and flanking sequences without the need to cut the PCR products with the restriction enzymes, which is typically the difficult step in PCR-mediated cloning. It also allows one to verify the sequences of the genes and create a Rif-r mutation in the rpoB genes in a small plasmid that does not express the subunit. Once the genes have been validated, they may then be transferred to pRNAPexpress, a variant of pIA423 (FIG. 3) in which the relevant restriction endonuclease sites flank the rpo gene locations.

The RNAP overexpression plasmid, for instance encoding B. anthracis RNAP and S. pneumoniae RNAP, may then be transferred to a suitable E. coli strain and tested for expression upon addition of IPTG to cultures. The presence of tags at the C-terminal end of the beta′ subunit (i.e. the rpoC gene product) allows ready identification of the xenogeneic RNAP in E. coli. For instance, a hexahistidine tag may be placed in this location (Anthony, et al. (2000) Protein Expr Purif 19, 350-354; Bushnell, et al. (2002) Proc Natl Acad Sci USA 99, 1218-1222. The xenogeneic RNAP can then be adsorbed to Ni²⁺-NTA agarose and the presence of all four subunits then verified by electrophoresis of eluent from the Ni²⁺-NTA agarose on denaturing polyacrylamide gels.

One possible complication in this example is the possibility that subunits from the xenogeneic RNAP may mix with subunits of E. coli RNAP during assembly. However, such subunit mixing was not observed when T. aquaticus RNAP was expressed in E. coli (Minhakin et al., 2001). Because inhibitor targets on RNAP will almost certainly be on the beta or beta′ subunits (the locations of all known inhibitor binding sites), the only complication for purposes of the invention will be mixing of E. coli and xenogeneic beta and beta′ subunits in a functional RNAP. If this problem is experienced, it can be eliminated by fusing the genes for the xenogeneic beta and beta′ subunits on the pRNAPexpress plasmid. The present inventors have found previously that such fusions of beta and beta′ yield functional RNAP enzymes containing only the fused polypeptide (Severinov, et al. (1997) J Biol Chem 272, 24137-24140.). As described in Example 1, should any of the culture components prove to inhibit the reporter protein, the cells may be washed free of culture medium and the added compounds prior to measuring fluorescence. Alternatively, the GFP fluorescence may be measured on a single-cell level using a fluorescence microscope. he cells may first be rinsed prior to activation of the reporter.

### Example 3

### Screening for Inhibitors of a Bacterial RNAP in a Bacterial Cell

The plasmid constructs described in Examples 1 and 2 can be used to screen for inhibitors of the expressed bacterial RNAPs in a bacterial culture. The overall approach is outlined in FIG. 4. In brief, cells containing the desired plasmids are grown in the presence of selection for the plasmids (in this case, ampicillin and kanamycin). Once the cells have reached early log-phase growth, IPTG (final concentration 1 mM) is added to the culture to induce overexpression of the plasmid-encoded RNAP subunits. When cultures reach mid-log growth, both rifampicin and the putative inhibitors are added; the former inhibits transcription by the wild-type RNAP; the latter, by the test RNAP. In certain cases, a range of concentrations of putative inhibitor is tested by adding different amounts to different mid-log cultures. Anhydrotetracycline is then added to induce the reporter gene. Expression of the reporter gene is monitored by placing the culture in a spectrofluorometer; in the event that inhibitors or antibiotics interfere with accurate reading of reporter gene expression, the cells are first washed to remove the interfering compounds.

An alternative method is to provide genes encoding a putative inhibitor. For example, BAC libraries obtained from diverse organisms can be transformed into the E. coli cell containing the two-plasmid test system, provided that the BAC library is constructed using plasmids compatible with those already present in the cells. BAC libraries may be constructed in a number of ways, including as described in WO0181567A2, herein incorporated by reference. As described in WO0181567A2, screening pools of library plasmids allows ultimate identification of the gene encoding the putative inhibitor. This approach enables the identification of previously unknown bacterial RNAP inhibitors.

FIG. 6 illustrates exemplary results expected from such experiments. Omission of IPTG, Tet, Rif, and inhibitors results in failure to produce GFP in both the test and control (Rif^{r} E. coli RNAP). Similarly, omission of IPTG and inhibitor in the presence of Tet and Rif does not yield GFP in either strain. Inclusion of IPTG, Tet, and Rif in the absence of inhibitor causes GFP to be produced in both strains. Inclusion of IPTG, Tet, Rif and an inhibitor specific for the test strain and not E. coli RNAP causes GFP to be produced only in the control strain, whereas inclusion of a generalized RNAP inhibitor prevents GFP production in either strain, as does an inhibitor of GFP.

### Example 4

### Screening for Inhibitors of a Eukaryotic RNAPII Subunit in a Eukaryotic Cell

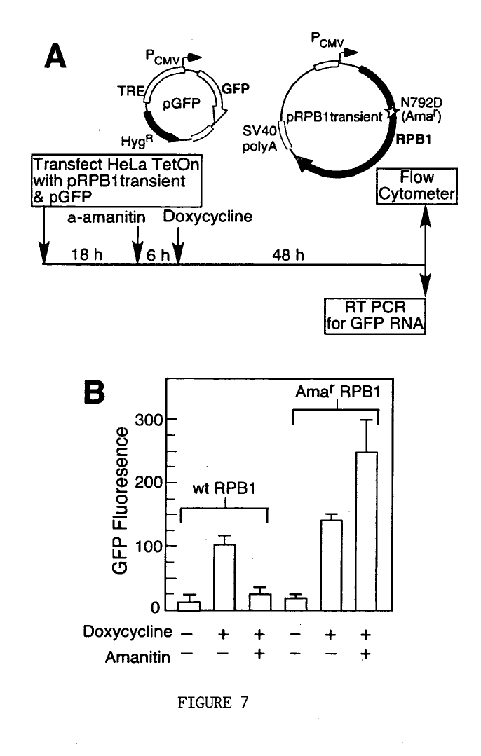

The method described here also can be adapted to identify inhibitors of eukaryotic RNAPII when expressed in eukaryotic cells. Such inhibitors would be effective poisons for mammalian species with actions similar to the known RNAPII inhibitor alpha-amanitin, but could be useful antibiotics in the case of eukaryotic pathogens. In this embodiment, the largest subunit of RNAPII, RPB1, or a fusion of the two largest RNAPII subunits, RPB1 and RPB2, is expressed from an strong mammalian promoter on a plasmid DNA. FIG. 7 illustrates an example of such an expression plasmid, pRPB1 express, which expresses the human RPB1 subunit from the CMV promoter. The function of the plasmid-expressed RPB1 or RPB1::RPB2 fusion subunit is specifically detected by the inclusion of an amanitin-resistance substitution in the RPB1 subunit and the addition of alpha-amanitin to the cell sample after the recombinant RNAPII subunits have been expressed. In this example, the N792D substitution is used to confer amanitin-resistance; any substitution that confers resistance to an inhibitor of RNAPII could be used in concert with the inhibitor in this assay. An extensive list of suitable amanitin-resistance substitutions is given in Bushnell et al. (2002) Proc Natl Acad Sci USA 99, 1218-1222.

The reporter in this method is expressed from a second plasmid under a regulated promoter such as the Tet-ON or Tet-OFF systems from Invitrogen. pGFP (FIG. 7A), which expresses a green fluorescent protein from the Tet-ON regulated CMV promoter is an example of such a reporter plasmid. Any reporter suitable for expression and detection in eukaryotic cells, such as but not limited to luciferase, beta-galactosidase, and beta-glucuronidase, can be used for this method. Any regulated promoter specific for eukaryotic RNAPII is suitable for this method.

To implement this method, eukaryotic cells (human HeLa or 293 cells in this example), are transfected with two plasmids, one of which encoded the RNAPII subunit (pRNAPexress in this example) and the other of which encodes a reporter expressed from an inducible promoter (PGFP in this example). The resident RNAPII is then inhibited by addition of amanitin and the function of the recombinant, amanitin-resistant RNAPII is detected by induction of the reporter, in this case induction of the GFP by addition of the tetracycline analogue doxycycline. After allowing appropriate time for expression of the reporter, 48 hours in this example, the reporter is detected for instance by passage through a flow cytometer. As is seen in FIG. 7B, the expression of functional RPB1 subunit from the plasmid results in fluorescent signal after addition of amanitin, whereas no fluorescent signal is observed when there is no amanitin resistant RPB1 expressed or when the reporter is not induced. Thus, the function of the resident RNAPII can be detected when amanitin is not added.

To screen for RNAPII inhibitors, candidate compounds are added to the assay after expression of the recombinant RNAPII subunit but before the induction of the reporter. Any compound that inhibits the RNAPII will eliminate production of the reporter signal. This method can be made specific for any xenogeneic RPB1 or RPB2 subunits that can combine with the resident RNAPII subunits to produce a functional chimeric RNAPII. In this situation, compounds that inhibit the chimeric RNAPII can be counterscreened in the same assay by omitting alpha-amanitin and using cells that do not express recombinant RNAPII. Compounds that inhibit RNAPII for eukaryotic pathogens without adverse affect on human RNAPII may be found by this method when the chimeric RNAPII includes RPB1 or RPB2 from the eukaryotic pathogen. Simultaneous screening for chimeric RNAPII-specific inhibitors can be accomplished by mixing cells containing the chimeric RNAPII and inhibited with amanitin with cells that express a distinguishable reporter such as a differently colored fluorescent protein prior to reading the signals.

### Example 5

### Screening for Inhibitors of a Bacterial or Bacteriophage RNAP in a Eukaryotic Cell

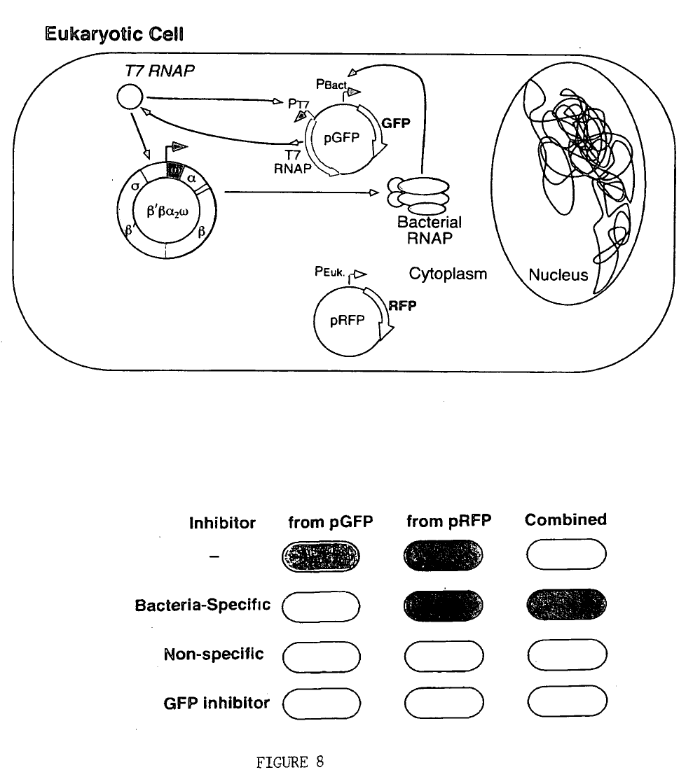

In some cases it is desirable to seek inhibitors of bacterial or bacteriophage RNAPs within eukaryotic cells. For instance, it may be desirable to find inhibitors that survive uptake into human cells where they can act on bacterial pathogens that penetrate and live inside human cells. This method can be used to screen for such inhibitors by using a pRNAPexpress plasmid (FIG. 5) that has been adapted for expression using T7 RNAP similarly to the expression of pIA423 (FIG. 4) and a reporter such as pGFP that is recognized by a bacterial RNAP. In this case, as illustrated in FIG. 8, the bacterial RNAP must either be fused to an appropriate sigma factor (Mooney, et al. (2003) Genes Dev 17, 2839-2851) or the sigma factor must be independently expressed and able to combine with the bacterial RNAP in order to direct transcription of the reporter gene. T7 RNAP can be expressed either from a third plasmid or can be included with pRNAPexpress or pGFP and is also introduced during transfection to initiate synthesis of the desired mRNAs. Alternatively, T7 RNAP may be produced from a gene located in a chromosome of the eukaryotic cell. The mixture of plasmids (and T7 RNAP when needed) is transfected into the eukaryotic cell and the synthesis of the bacterial RNAP is directed by expression of T7 RNAP, which transcribes the bacterial RNAP genes. Expression of the reporter can be induced after a suitable interval and addition of an inhibitor of the resident RNAP with a compound such as amanitin, if necessary. Alternatively, the reporter can be constitutively expressed provided that its expression is solely dependent on the presence of functional bacterial RNAP. In this case, as illustrated in FIG. 8, inclusion of a distinguishable reporter that is specific for the resident eukaryotic RNAPII (PRFP here encoding a red fluorescent protein) can also be introduced and will allow ready discrimination of compounds that inhibit the bacterial RNAP without affecting the eukaryotic RNAPII. Precedent exists for expression of genes from plasmids using T7 RNAP in the cytoplasm of transfected mammalian cells as described in Chen et al. (1994) Nucleic Acids Res. 22, 2114-21220.

In another implementation of this assay, it is possible to combine eukaryotic and prokaryotic cells in which expression of reporters depends on the same or different RNAPs to screen for compounds that have particular sets of desired properties. For instance, one could screen for compounds that inhibit a particular bacterial RNAP in a bacteria while not inhibiting another RNAP, either bacterial RNAP or a eukaryotic chimeric RNAP, within a eukaryotic cell.

### Example 6

### Additional Demonstration of Conditional Expression of a Reporter Gene by E. coli RNAP Over Expressed from a Plasmid

Reporter gene constructs in which expression of a reporter protein is under control of the Tet repressor may be constructed as follows. The reporter protein (at least for the purposes of this example) will be the green fluorescent protein (GFP) from the jellyfish A. victoria, a readily available research tool. Several properties of Tet repressor make it the preferred expression system. It is a simple bacterial repressor that tightly regulates promoters when its operator site is located within a promoter sequence. It can be readily supplied to bacteria without unwanted side-effects by expression from a copy of the tet repressor gene integrated in the bacterial chromosome. Finally, and most importantly, it regulates transcription initiation at a promoter in a simple manner, such that addition of an inducer will allow transcription to occur without the need for additional protein synthesis.

As illustrated in FIG. 2, a TetR-controlled promoter sequence may be inserted upstream from a selected GFP gene. The promoter DNA may be synthesized using oligonucleotides ligated upstream from a promoter-less GFP gene in a plasmid that contains the TetR gene transcribed in the opposite direction. This entire cassette may either be maintained on a plasmid or transferred to the chromosome of E. coli by recombination to yield a stable integrant. Either method allows the tet repressor-regulated reporter be readily moved from strain to strain by plasmid transformation or phage P1 transduction of the integrated reporter.

To obtain an RNAP overexpression plasmid suitable for use in the invention, a previously-described plasmid, pIA423 (Artsimovitch et al., 2003, FIG. 3) may be utilized. pIA423 expresses E. coli RNAP under control of the lac repressor (LacR; encoded by lacIQ). To obtain a plasmid that expresses a version of RNAP that can be selected by addition of rifampicin (Rif) to cultures, a mutation encoding the Rif-resistance mutation rpoB (S531F) may be introduced into the rpoB gene of pIA423. This manipulation can be accomplished readily by standard techniques (e.g., Landick et al., 1990). The resulting plasmid will then express a Rif-resistant RNAP only when induced by addition of IPTG to bacterial cultures. Alternatively, if a plasmid-encoded rpoB gene product combines with other RNA polymerase subunits encoded in the E. coli chromosome to yield a functional RNA polymerase, the rpoB gene alone bearing the S531F mutation may be expressed from an IPTG-regulated plasmid such as pRL702 (Artsimovitch et al, 2003). To detect inhibitors of other bacterial RNA polymerases, the relevant RNA polymerase subunit genes may be expressed from a pIA423-like plasmid or, if the rpoB gene alone functions with E. coli subunit genes and is a desired target for inhibitor screens, expressed from a pRL702-like plasmid.

The Rif^{r}-RNAP overexpression plasmid may then be combined with the GFP-reporter strain and tested for specific expression of the reporter by the plasmid-encoded RNAP. Construction of this test strain is according to transformation techniques known in the art. Steps depicted in FIG. 4 may then be carried out using the thusly-constructed strain. First, the strain is grown in liquid culture to early log phase. Expression of Rif^{r}-RNAP is then induced by adding IPTG. After continuing growth long enough to let the RNAP accumulate, Rif is then added to the culture to inhibit the wild-type RNAP present in the cell. Anhydrotetracycline or a non-toxic level of tetracycline (0.2 μg/ml) is then added to remove TetR from the promoter in front of the GFP reporter gene in the cell. Since the wild-type cellular RNAP will be inhibited, only the RNAP made from the overexpression plasmid, which is resistant to Rif, will be able to make mRNA from the reporter gene. The appearance of GFP in the culture is then monitored using a spectrofluorimeter at the emission wavelength for the respective GFP reporter.

Two potential complications in the above-described scheme may be avoided as follows. First, the culture medium, Rif, anhydrotetracycline, or tetracycline may interfere with detection of GFP fluorescence. If this happens, the cells may be washed free of culture medium and the added compounds prior to measuring fluorescence. Alternatively, the GFP fluorescence may be measured on a single-cell level using a fluorescence microscope. A second complication may arise if Rif fails to inhibit the cellular RNAP. This occurrence may be controlled for by using a Rif-sensitive version of the RNAP overexpression plasmid. In this case, Rif should completely eliminate the appearance of GFP in the experimental procedure set forth in FIG. 4.

A specific illustration of this method was achieved using the pRM547 derivative (SEQ ID NO:1) of the pRL702 rpoB (S531F) plasmid in combination with a GFP reporter plasmid called pAI8 (SEQ ID NO:3). pRL702rpoB(S531F) is equivalent to pIA178 in Artsimovitch et al, 2003b except lacking the silent XhoI site present in pAI178. pRM547 was constructed from pRL702rpoB(S531F) by replacement of the 1.8 kb NheI-ScaI fragment containing the ampicillin-resistance gene and ColE1 replication origin with a 1 kb XbaI-NruI fragment containing the chloramphenicol-resistance gene and p15 origin from pACYC184 (Chang, et al. (1978) J Bacteriol 134(3):1141-1156). As a control, pRM546 (SEQ ID NO:2) was constructed similarly from pRL702 (Artsimovitch et al, 2003) to have the chloramphenicol-resistance gene and p15 origin but with the rif-sensitive (wildtype) rpoB gene instead of the rif-resistant (S531F) rpoB. pAI8 was constructed by first recovering the gfp+ gene from pWH1012gfp+ (Schlotz et al., 2000) on a DNA fragment containing BsaI restriction sites upstream and downstream of the gfp+ gene (gfp+ is a mutated version of GFP with improved fluorescence properties that is described in Scholtz, et al. (2000) Eur. J. Biochem. 267, 1565-1570.). This fragment was made by PCR using the following two oligonucleotide primers follow by digestion with BsaI. The upstream primer was 5′-CACACGGTCTCNAATGGCCAGCAAAGGAGAAGAACTTTTCAC (SEQ ID NO:6). The downstream primer was 5′-CACACAAGCTTACTTGTACAGCTCGTCCATGCC (SEQ ID NO:7). The digested fragment was ligated between into pASK-IBA3plus that was digested with BsaI and HindIII. pASK-IBA3plus is a commercially available plasmid that expresses Tet repressor and carries the tetA promoter that is regulated by Tet repressors, the ampicillin-resistance gene, and the ColEI replication origin (pASK-IBA3plus is obtained from IBA GmbH of Gottingen, Germany through its distributor in St. Louis, Mo. (www.igo-go.com). Because pRM546 and pRM547 encode choramphenicol-resistance and use the p15 replication origin and pAI8 encodes ampicillin-resistance and uses the ColE1 replication origin, pAI8 can be stably maintained in E. coli cells simultaneously with either pRM546 or pRM547. In an alternative configuration, the reporter plasmid can be constructed to contain the p15 origin and encode chloramphenicol resistance, as in pAI9 (SEQ ID NO:4), and carried in a cell comprising a compatible ColE1 plasmid encoding rpoB. Finally, this alternative configuration can be reconfigured so that the reporter plasmid expresses GFP from an arbitrary promoter sequence that is optimized for a given RNA polymerase or sigma factor by ligating synthetic oligonucleotides specifying the promoter sequence between the SphI and BglII restriction sites of pAI10 (SEQ ID NO:5). pAI10 is a derivative of pAI9 in which the tetA promoter has been replaced by a promoterless SphI-BglII DNA cassette.

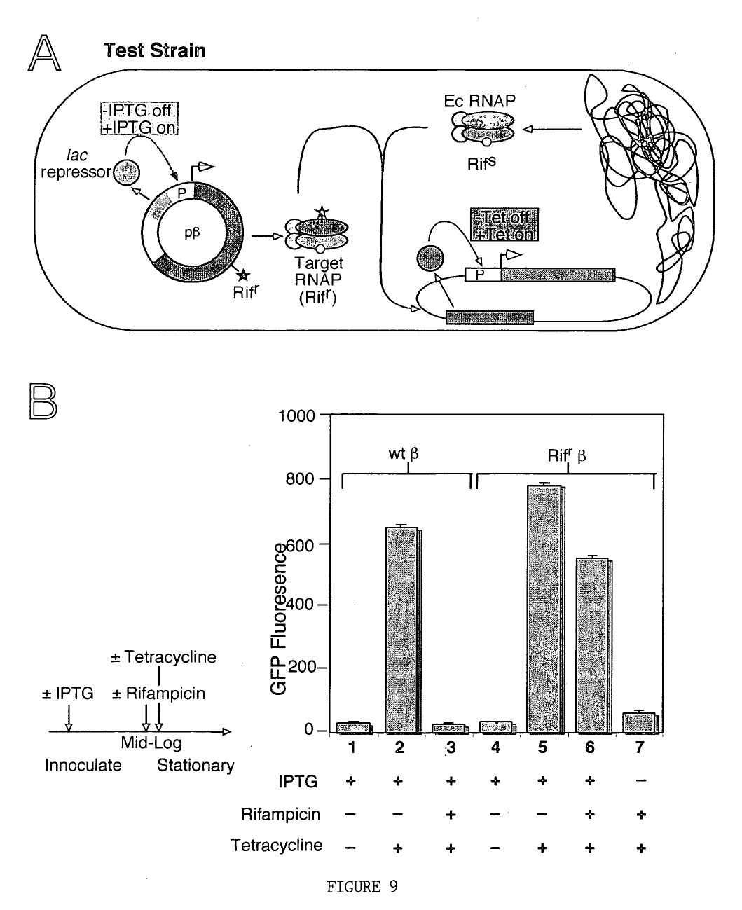

To detect GFP expression dependent on a plasmid-encoded RNA polymerase subunit, pRM546 and pRM547 were individually combined with pAI8 by sequential transformation into the E. coli strain DH5alpha (Hanahan D (1983) J Mol Biol 166: 557-580). The strains were grown with shaking at 37° C. in LB medium plus 34 μg chloramphenicol/ml and 50 μg ampicillin/ml. To induce RNA polymerase subunit expression, IPTG was added to 1 mM at the earliest point that the presence of bacteria can be detected in the culture visually (corresponding to 2-5 density units measured on a Klett-Summerson colorimeter; “Klett units”). The bacteria were then allowed to grow until they reached a density of 25-30 Klett units. To inhibit the chromosomally encoded RNAP, rifampicin was added to 50 μg/ml final concentration and growth was continued for 15 minutes, at which point tetracycline was added to 0.2 μg/ml to those cultures in which GFP induction was desired. Growth was then continued for 1 hour. The cells were then recovered by centrifugation at 5000×g for 10 min. After careful removal of all supernatant, the cell pellets were individually resuspended in M9 buffer (6 g Na₂HPO₄, 3 g KH₂PO₄, 0.5 g NaCl, 1 g MH₄Cl per liter H₂O) and adjusted to a density equivalent to absorbance 0.1 at 600 nm as measured in a spectrophotometer (CD₆₀₀). These samples were then read in a spectrofluorimeter with excitation at 491 nm and emission at 512 nm to record fluorescence, and the readings were converted to fluorescence units per CD₆₀₀. Typical results are illustrated in FIG. 9B.

The results of this assay clearly establish the robust nature of the screening assay. GFP is detected only when tetracycline inducer is added and when expression of a plasmid-encoded copy of the rpoB(S531F; Rif^{r}) subunit has been induced by IPTG (FIG. 9B, column 6) or when rifampicin is not added to the culture (FIG. 9B, columns 2 and 5). No significant amount of GFP is detected when IPTG is not added to induce expression of the rpoB(S531F; Rif^{r}) subunit (FIG. 9B, column 7), when GFP expression is not induced (FIG. 9B, columns 1 and 4), or when the plasmid-borne copy of the rpoB gene lacks the S531F-Rif^{r} mutation (FIG. 9B, column 3). The level of GFP fluorescence produced when GFP expression depends on the plasmid-encoded copy of the rpoB(S531F; Rif^{r}) subunit is high enough that it is readily visible to the naked eye when the resuspended bacteria are exposed to 365 m ultraviolet light on a trans-illuminator. Thus, the level of GFP expression is ample for adaptation of the assay to a multi-well plate reader and high-throughout screening for inhibitors of the plasmid encoded RNA polymerase.

A variant of the assay is possible that avoids the use of rifampicin to inhibit the cellular RNA polymerase through use of strains with temperature sensitive mutations that inactivate the cellular RNA polymerase. For instance, a mutation in the beta′ subunit of E. coli RNA polymerase that changes Gly1360 to Asp (rpoC1) blocks RNA polymerase activity at 42° C., as does the rpoC397 mutation that replaces 52 amino acids distal to position 1355 with 23 unnatural residues (Gross, et al. (1976) Mol. Gen. Genet. 147:337-341; Gross, et al. (1977) Eur. J. Biochem. 81:333-338; Christie, et al. (1996) J. Bacteriol. 178:6991-6993; Nedea, et al. (1999) J. Bacteriol. 181:2663-2665). Thus, instead of adding rifampicin to inhibit the endogenous RNA polymerase, the use of strains bearing temperature-sensitive RNA polymerase mutations like rpoC1 and rpoC397 that block RNA polymerase activity after a temperature shift will allow inhibition of endogenous RNA polymerase by temperature shift prior to the induction of the GFP reporter by addition of an inducer of the Tet repressor.

This method can be generalized to any cellular RNA polymerase, eukaryotic, archaeal, or prokaryotic, because a mutation in eukaryotic RNA polymerase II in Saccharomyces cerevisiae at the position homologous to the G1360D rpoC1 mutation (called rpb1-1 in Saccharomyces cerevisiae RNA polymerase II and causing the G1437D substitution in the RPB1 subunit; Scafe, et al. (1990) Mol. Cell. Biol. 10: 1270-1275) exhibits the same property of inactivating cellular RNA polymerase II upon temperature shift. Since RNA polymerase II is evolutionarily distant from E. coli RNA polymerase, the ability to produce temperature sensitive enzyme activity by a substitution at this position should be a universal property of multisubunit RNA polymerases in all bacteria, archaea, and eukaryotes that contain this conserved residue. For the purpose of this method, any mutation that confers temperature-sensitive activity on RNA polymerase or any other condition-dependency on RNA polymerase activity will be applicable to inhibit an endogenous RNA polymerase and allow assay of a foreign RNA polymerase by subsequent induction of a reporter.

Those skilled in the art will recognize, or be able to ascertain using no more then routine experimentation, numerous equivalents to the specific methods, assays and reagents described herein. Such equivalents are considered to be within the scope of this invention and covered by the following claims. All publications, patents, and patent applications cited herein are hereby incorporated by reference in their entirety for all purposes.

What is claimed is:

1. An in vivo screening method for identifying an inhibitor of a ribonucleic acid polymerase (RNAP), comprising steps of:
(a) expressing a test RNAP in a cell containing a first reporter;
(b) expressing a control RNAP in the cell of step (a) that also contains a second different reporter or, alternatively, a different cell that contains the second different reporter, wherein said first and second different reporters distinguish activities of the test and control RNAPs, respectively;
(c) contacting the cell or cells of step (b) with a candidate compound; and
(d) assaying to obtain a combined signal from the first reporter and second different reporter wherein a unique combined signal identifies the candidate compound as an inhibitor of an RNAP.

2. The method according to claim 1 wherein the inhibitor identified by the method is a specific inhibitor of the test RNAP and selective inhibition of the test RNAP relative to the control RNAP is indicated by the unique combined signal.

3. The method according to claim 1 wherein the inhibitor identified by the method is a general inhibitor of the test RNAP and control RNAP and general inhibition is indicated by the unique combined signal.

4. The method according to claim 1 wherein activities of the test and control RNAPs are distinguished from each other by the first and second different reporters which are selectably-transcribed by the test and control RNAPs, respectively.

5. The method according to claim 1 wherein the test and control RNAPs are drug resistant and their activities are further distinguished from activities of a host RNAP also contained within the cell or cells by the addition of a drug which inhibits said host RNAP but not said drug resistant test and control RNAPs.

6. The method according to claim 5 wherein said drug resistant RNAPs are resistant to α-amanitin.

7. The method according to claim 5 wherein said drug resistant RNAPs are resistant to streptolydigin.

8. The method according to claim 5 wherein said drug resistant RNAPs are resistant to rifampicin.

9. The method according to claim 8 wherein said drug resistant RNAPs independently comprise a Ser531 to Phe or Asp514 to Val mutation in an RNAP beta subunit.

10. The method according to claim 1 wherein said candidate compound is expressed from a nucleic acid construct present within said cell or cells of step (b).

11. The method according to claim 1, wherein said cell or cells of step (b) are bacteria.

12. The method according to claim 1 wherein said cell or cells of step (b) are eukaryotic cell or cells.

13. The method according to claim 1 wherein said first reporter and said second different reporter are differently-colored fluorescent proteins.

14. The method according to claim 1 wherein expression of said test and control RNAPs are independently under control of an inducible promoter.

15. The method according to claim 14 wherein said inducible promoter is under control of lacR.

16. The method according to claim 1 wherein said first reporter and said second different reporter are independently under control of inducible promoters.

17. The method according to claim 16 wherein said inducible promoters are under control of tetR.

18. The method according to claim 1 wherein said test RNAP and said control RNAP represent xenogeneic RNAPs.

19. The method according to claim 1 wherein said test RNAP and said control RNAP represent variants of the same naturally-occurring RNAP.

20. The method according to claim 1 wherein said cell or cells include a temperature sensitive host RNAP and activities of the test and control RNAPs are distinguished from activities of said temperature sensitive host RNAP upon inactivation of said host RNAP by temperature shift.
